SEQUENCE LISTING (6 sequences)

SEQ ID NO: 1 - DNA, 1809 bp, Homo sapiens
  source 1..1809: mol_type = genomic DNA
atgcatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggatcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaa

SEQ ID NO: 2 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 3 - DNA, 1809 bp, Homo sapiens
  source 1..1809: mol_type = other DNA
atgcatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtcactttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggatcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtccccaaccacatgctgttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctctaa

SEQ ID NO: 4 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGHFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPNHMLLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 5 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 6 - protein, 574 aa, Homo sapiens
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGHFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPNHMLLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

(19) United States
(12) Patent Application Publication
(10) Pub. No.: US 2026/0077059 A1
(43) Pub. Date: Mar. 19, 2026

(54) ANTI-GHRELIN BUTYRYLCHOLINESTERASE ENZYME THERAPEUTIC

(71) Applicant: BATTELLE MEMORIAL INSTITUTE, Columbus, OH (US)

(72) Inventor: Matthew Neal, Columbus, OH (US)

(21) Appl. No.: 19/329,487

(22) Filed: Sep. 15, 2025

Related U.S. Application Data
(60) Provisional application No. 63/695,692, filed on Sep. 17, 2024.

(51) Int. Cl. A61K 48/00; A61K 9/51; A61K 38/00; C12N 9/18; C12N 15/85; C12N 15/88

(52) U.S. Cl. CPC A61K 48/005; A61K 9/5138; C12N 9/18; C12N 15/85; C12N 15/88; A61K 38/00; C07K 2319/60; C12Y 301/01008

(57) ABSTRACT

Compositions and methods are provided for inactivating ghrelin in mammalian cells by administration of a polynucleotide encoding a butyrylcholinesterase (BChE) enzyme.

### CROSS REFERENCE TO RELATED APPLICATIONS

This non-provisional application claims the benefit and priority, under 35 U.S.C. § 119 (e) and any other applicable laws and statutes, to U.S. Provisional Application Ser. No. 63/695,692 filed on Sep. 17, 2024, the entire disclosure of which is incorporated herein by reference.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as a 12,000 byte xml file named “428194.xml,” created on Aug. 22, 2025.

### BACKGROUND

Ghrelin is a hormone primarily produced by enteroendocrine cells of the gastrointestinal tract, especially the stomach, however, ghrelin crosses the blood-brain barrier and communicates with central rewarding systems. Ghrelin is known to participate in regulation of reward cognition, learning and memory, the sleep-wake cycle, taste sensation, reward behavior, and glucose metabolism. Ghrelin also activates cells in the anterior pituitary gland and hypothalamic arcuate nucleus, including neuropeptide Y neurons that initiate appetite.

Ghrelin only becomes active when caprylic (octanoic) acid is linked posttranslationally to serine at the 3-position by the enzyme ghrelin O-acyltransferase (GOAT) to form a proteolipid. The signaling from activated Ghrelin acts 1) as a hunger signal to get a person to eat, 2) to increase adipogenesis, or generation of fat cells, 3) to increase insulin resistance, leading to hyperglycemia and Type II diabetes, and 4) to increase addiction cravings for everything from alcohol to drugs and beyond. It has been shown that inhibition of ghrelin signaling has led to weight loss, and increased insulin sensitivity. The non-octanoylated form is desacyl ghrelin, and it does not activate the GHS-R receptor.

Butyrylcholinesterase (BChE) is a nonspecific cholinesterase enzyme found in all mammals that is synthesized by the liver and secreted into the circulation. Recent findings point toward a physiological role in hydrolyzing the appetite-promoting hormone, ghrelin. In particular, BChE has been shown to cleave the n-ocanoyl group and therefore inactivating ghrelin.

### SUMMARY

The present disclosure is directed to compositions and methods for inactivating ghrelin in vivo by administering to a mammal a polynucleotide that encodes butyrylcholinesterase (BChE). More particularly, in one embodiment a polynucleotide is provided that expresses a BChE polypeptide having an enhanced ability to hydrolyze acyl ghrelin (thus inactivating ghrelin) relative to native human BChE (SEQ ID NO: 2). In one embodiment the polynucleotide encodes a polypeptide comprising SEQ ID NO: 4 or 6. In one embodiment the polynucleotide encodes a polypeptide comprising SEQ ID NO: 5 modified to comprise one or more amino acid substitutions selected from G117H, Y282N, G283H, T284M, and P285LG117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment the polynucleotide encodes a polypeptide comprising SEQ ID NO: 6 modified to comprise one or more amino acid substitutions selected from A199S, S226A, S287G, A328W, F329M, and Y332G (wherein the amino acid positions are numbered with respect to SEQ ID NO: 6). In one embodiment the polynucleotide encoding butyrylcholinesterase (BChE) comprises the sequence of SEQ ID NO: 3.

In one embodiment the polynucleotide encoding BChE is operably linked to regulatory elements that allow for the expression of the polynucleotide in mammalian cells. In one embodiment an expression cassette comprises a nucleic acid sequence that encodes a polypeptide of SEQ ID NO: 5 modified to comprise one or more amino acid substitutions selected from G117H, Y282N, G283H, T284M, and P285LG117H, Y282N, G283H, T284M, and P285L, and one or more amino acid substitutions selected from A199S, S226A, S287G, A328W, F329M, and Y332G wherein the polynucleotide is operably linked to a promoter that functions in mammalian cells. In one embodiment the promoter is an inducible promoter that is functional as an inducible promoter in mammalian cells. In one embodiment the promoter is a Cytomegalovirus (CMV) promoter, optionally paired with a tetracycline regulatory element (TRE). In one embodiment a plasmid is provided comprising the expression cassette.

In one embodiment a composition is provided comprising a delivery vehicle and a nucleic acid sequence that expresses any one of the BChE disclosed herein, including a polynucleotide encoding a modified peptide of SEQ ID NO: 5 or 6 as disclosed herein, wherein the nucleic acid sequence is bound or encapsulated by the delivery vehicle. In one embodiment the delivery vehicle is a viral vector, including for example an adenovirus vector or an adeno-associated viral vector. In another embodiment the delivery vehicle is a non-viral polymeric nanoparticle delivery vehicle. In one embodiment the delivery vehicle can be selected or modified to comprise a targeting moiety that directs the delivery vehicle to a specific cell type, tissue or organ upon administration to a mammal.

In accordance with one embodiment a method of inactivating ghrelin in vivo is provided. The method comprises delivering a polynucleotide that encodes BChE to the cells of a mammal via a delivery vehicle, including for example a non-viral nanoparticle delivery vehicle. In one embodiment the non-viral nanoparticle delivery vehicle comprises RAFT block copolymers, as described in published US Patent no. 2022/0333097, the disclosure of which is incorporated herein in its entirety. In one embodiment the non-viral nanoparticle delivery vehicle comprises a nucleic acid sequence, including origami nucleic acid sequences, as described in published US Patent no. 2022/0175812 and 2024/0067960, the disclosures of which are incorporated herein in its entirety. In accordance with one embodiment a composition comprising the non-viral nanoparticle delivery vehicle and a polynucleotide that encodes a polypeptide comprising SEQ ID NO: 5 modified to comprise one or more amino acid substitutions selected from G117H, Y282N, G283H, T284M, and P285LG117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5) is administered to a mammal to induce overexpression of BChE in the cells of the mammal, wherein the overexpression of the BChE results in deacylation and inactivation of ghrelin. In one embodiment the polynucleotide encodes the polypeptide of SEQ ID NO: 6 or SEQ ID NO: 6 modified to comprise one or more amino acid substitutions selected from A199S, S226A, S287G, A328W, F329M, and Y332G (wherein the amino acid positions are numbered with respect to SEQ ID NO: 6).

The method of administering the non-viral nanoparticle delivery vehicle includes any of the standard routes, including via an intramuscular, an intravenous, an intraperitoneal, an oral, or a pulmonary route. In one embodiment the delivery vehicle can be targeted to a particular cell type, tissue or organ by selection of the surface charge, particle size, or by conjugation with various targeting moieties.

### BRIEF DESCRIPTIONS OF THE DRAWINGS

FIG. 1 provides the nucleic acid sequence of a wild-type human butyrylcholinesterase (SEQ ID NO. 1).

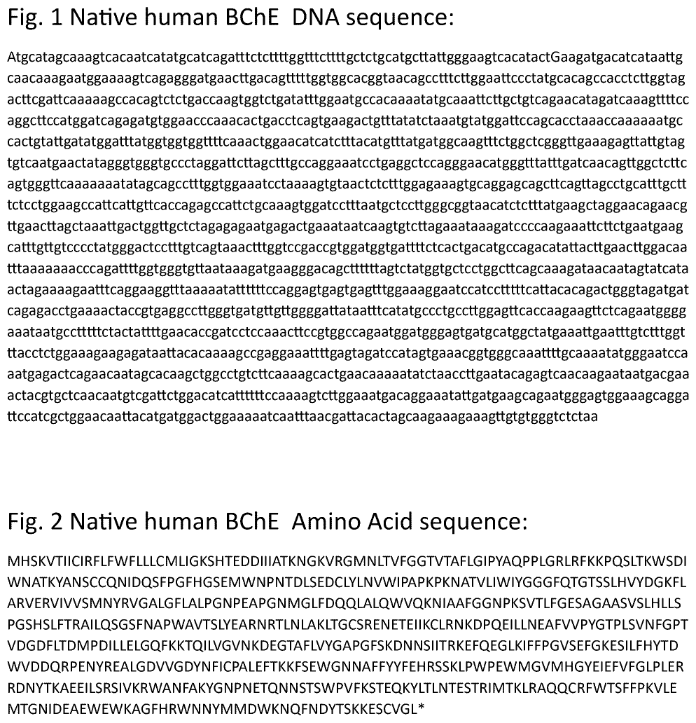

FIG. 2 provides the amino acid sequence of wild-type human butyrylcholinesterase (SEQ ID NO. 2).

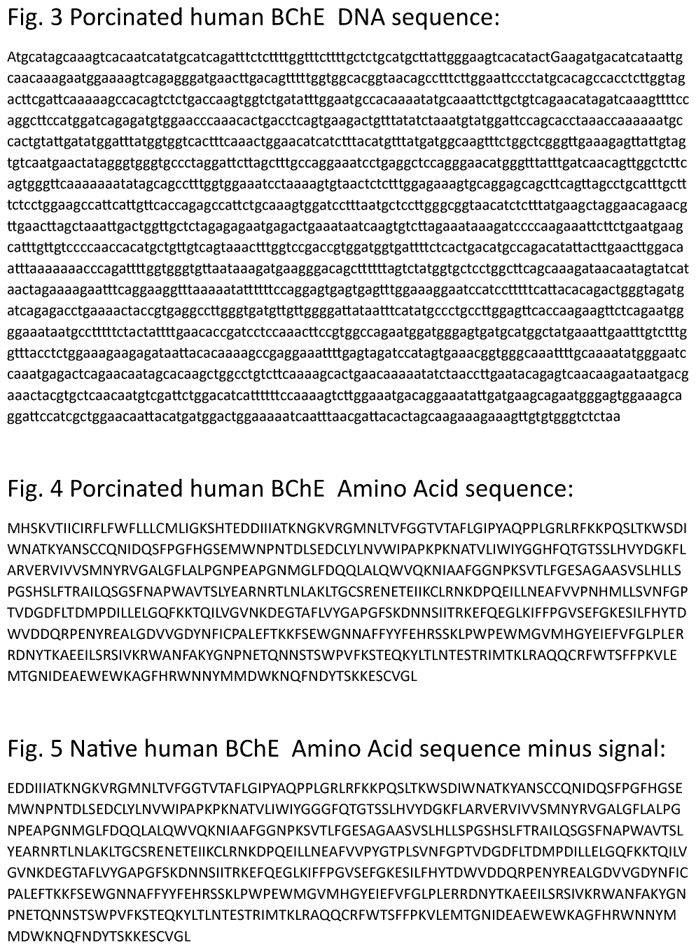

FIG. 3 provides the nucleic acid sequence of a porcinated human butyrylcholinesterase (SEQ ID NO. 3).

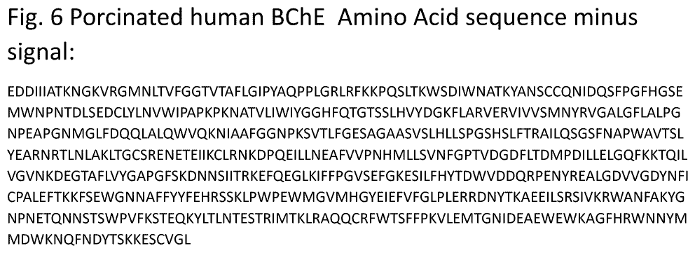

FIG. 4 provides the amino acid sequence (SEQ ID NO: 4) of the butyrylcholinesterase polypeptide encoded by the porcinated human butyrylcholinesterase of SEQ ID NO: 3.

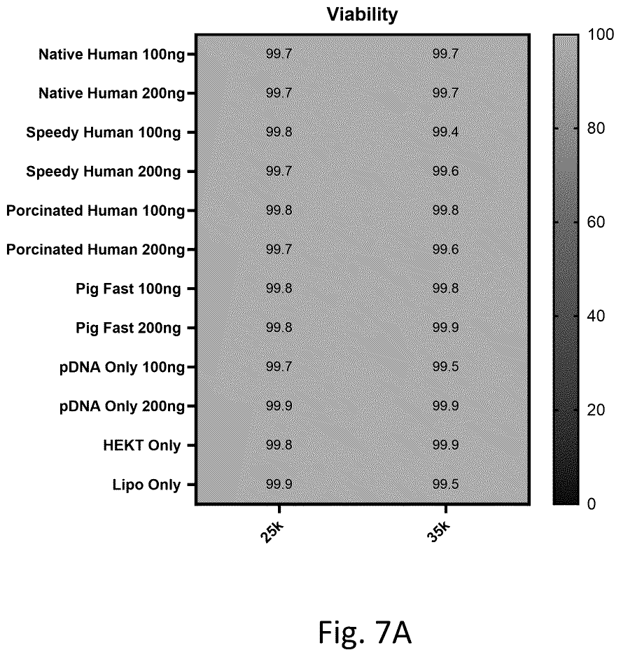

FIG. 5 provides the amino acid sequence of wild-type human butyrylcholinesterase with the N-terminal signaling peptide removed (SEQ ID NO: 5).

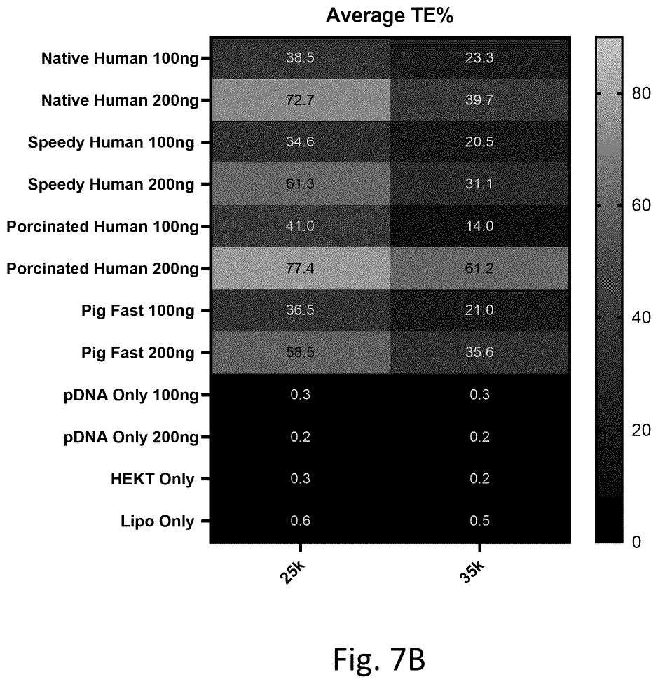

FIG. 6 provides the amino acid sequence of porcinated human butyrylcholinesterase with the N-terminal signaling peptide removed (SEQ ID NO: 6).

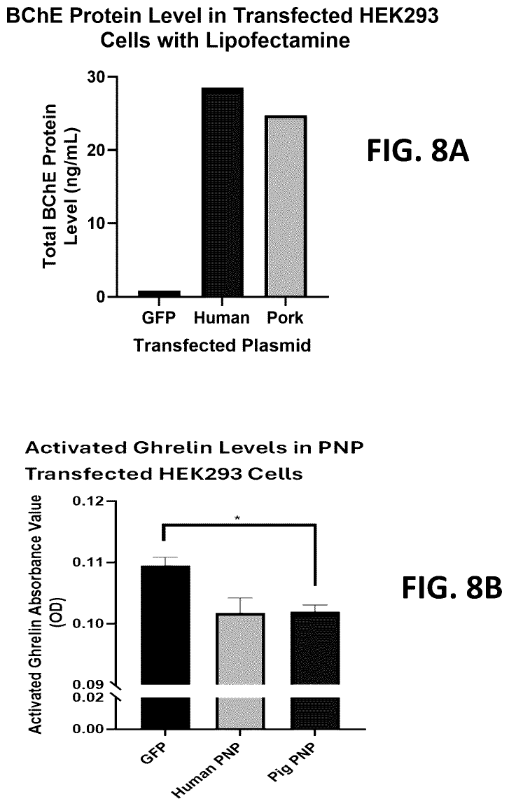

FIGS. 7A & 7B show the percent viability (FIG. 7A) of HEK cells 48 hours post transfection with human or porcinated butyrylcholinesterase (BChE) in various carriers, and the transfection efficiency (FIG. 7B) of the same transfections.

FIGS. 8A & 8B: HEK cells were transfected in vitro with a GFP reporter in Liofectamine, human BChE overexpression plasmid with a GFP reporter in Lipofectamine and porcinated human BChE overexpression with a GFP reporter. Following incubation with polymeric nanoparticles containing the nucleic acid cargo, transfection efficiency was determined by fluorescence imaging. Transfection efficiencies for two sets of experiments were as follows: CMV GFP: 51.4% and 34.6%; Human GFP: 42/0% and 38.7% and porcinated human GFP: 22.9 and 30.2%. FIG. 8A is a graph showing the results of a BChE protein ELISA conducted on the transfected cells demonstrating significant elevation in both human and porcinated BChE. FIG. 8B is a graph showing the results of an activated Ghrelin protein ELISA demonstrating reduction in activated ghrelin levels for administered porcinated BChE and human BChE.

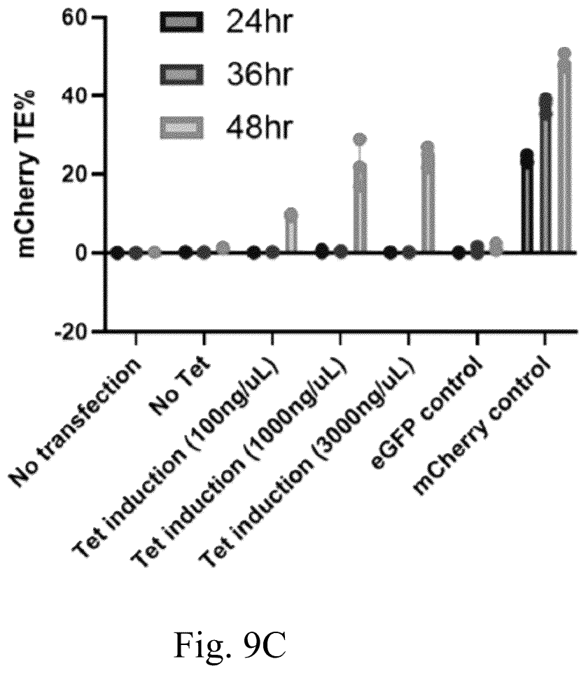

FIG. 9A-9D show the delivery and expression of BChE episomal DNA in cells as measured by fluorescence using a pEPI-TetON-pBChE-Crimson episome construct. FIG. 9A is a schematic map of the pEPI-TetON-pBChE-Crimson construct comprising the constitutive CMV promoter expressing GFP and the inducible Tet promoter expressing both BChE and the Crimson RFP separated by a cleavable P2A peptide. FIG. 9B shows the baseline transfection capabilities of the pEPI-TetON-pBChE-Crimson episome by way of eGFP fluorescence, wherein the transfection efficiency, via percentage of GFP positive cells following lipofectamine transfection is shown, at three different timepoints 24 hr, 36 hr and 48 hr. FIG. 9C shows the induction efficiency (via addition of doxycycline to the cultures) of the tetracycline response element (TRE) by way of Crimson (red). FIG. 9D is an image of eGFP expressing cells demonstrating longer duration of expression with the epiDNA (comprising the pEPI-TetON-pBChE-Crimson episome construct) compared to plasmid DNA (pDNA) transfected to the same cell type at the same time.

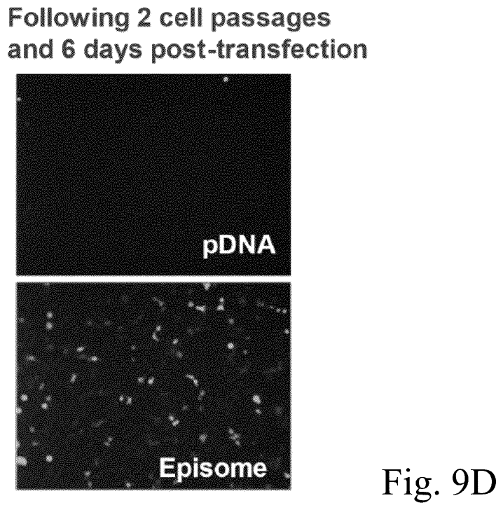

FIG. 10 provides an image of mice administered PNP injected via intramuscular route and imaged for fluorescent transfection using IVIS instrument.

### DETAILED DESCRIPTION

Before the present disclosure is further described, it is to be understood that this disclosure is not limited to any particular embodiment described herein.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

### Definitions

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values. To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Concentrations that are given as percentages refer to mass ratios, unless indicated differently.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring compound present in a living animal is not isolated, but the same compound, separated from some or all of the coexisting materials in the natural system, is isolated.

As used herein, the terms “including,” “containing,” and “comprising” are used in their open, non-limiting sense.

As used herein absent further modifying language, the term “porcinated BChE” refers to a polypeptide comprising the sequence of SEQ ID NO: 6.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein a “plasmid” is a circular, self-replicating piece of DNA that can replicate independently of the host cell.

As used herein the term “episome” absent further elaboration defines a special type of plasmid, which remains part of the eukaryotic genome without integration. Episomes replicate together with the rest of the genome and associate with metaphase chromosomes during mitosis.

As used herein the term “delivery vehicle” defines any composition of matter that can associated with a bioactive material and enhance localized concentrations of the bioactive agent to a target organ, tissue or cell type after delivery of the composition to an animal, including humans. The delivery vehicle can be a viral or non-viral delivery vehicle used to deliver polypeptides, or more typically polynucleotides, to the target organ, tissue or cells. Examples of non-viral delivery vehicles for delivering genetic material include Lipid Nanoparticles (LNPs), Polymer Nanoparticles (PNPs), Extracellular Vesicles (EVs), for example an exosome, polyplexes formed with cationic polymers (e.g., Poly(ethyleneimine) (PEI)), liposomes, inorganic nanoparticles (e.g., gold, silica), and cell-penetrating peptides.

As used herein a bioactive agent is “associated with a delivery vehicle” when the bioactive agent is bound to, or otherwise linked to, the delivery vehicle via covalent, ionic, or hydrogen boding or encapsulated or embedded in the structure of the delivery vehicle.

### EMBODIMENTS

In one embodiment a variant of human BChE is provided that comprises the amino acid of SEQ ID NO: 5 modified to comprise one or more amino acid substitutions selected from G117H, Y282N, G283H, T284M, and P285LG117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a variant of human BChE is provided that comprises a peptide of SEQ ID NO: 5 modified to comprise two more amino acid substitutions selected from G117H, Y282N, G283H, T284M, and P285LG117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a variant of human BChE is provided that comprises a peptide of SEQ ID NO: 5 modified to comprise three more amino acid substitutions selected from G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a variant of human BChE is provided that comprises a peptide of SEQ ID NO: 5 modified to comprise four or more amino acid substitutions selected from G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a variant of human BChE is provided that comprises a peptide of SEQ ID NO: 5 modified to comprise amino acid substitutions G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment the variant of human BChE comprises a peptide of SEQ ID NO: 6. In one embodiment the variant of human BChE comprises a peptide of SEQ ID NO: 6 further modified by the inclusion of one or more amino acid substitutions selected from A199S, S226A, S287G, A328W, F329M, and Y332G (wherein the amino acid positions are numbered with respect to SEQ ID NO: 6). In one embodiment the variant of human BChE comprises a peptide of SEQ ID NO: 4. In one embodiment a polynucleotide is provide that encodes any of the variant of human BChE peptides disclosed herein.

In one embodiment the variant of human BChE comprises a peptide of SEQ ID NO: 6 modified to comprise one or more amino acid substitutions selected from A199S, S226A, S287G, A328W, F329M, and Y332G (wherein the amino acid positions are numbered with respect to SEQ ID NO: 6). In one embodiment the variant of human BChE comprises a peptide of SEQ ID NO: 6 modified to comprise two or more amino acid substitutions selected from A199S, S226A, S287G, A328W, F329M, and Y332G (wherein the amino acid positions are numbered with respect to SEQ ID NO: 6). In one embodiment the variant of human BChE comprises a peptide of SEQ ID NO: 6 modified to comprise three more amino acid substitutions selected from A199S, S226A, S287G, A328W, F329M, and Y332G (wherein the amino acid positions are numbered with respect to SEQ ID NO: 6). In one embodiment the variant of human BChE comprises a peptide of SEQ ID NO: 6 modified to comprise four or more amino acid substitutions selected from A199S, S226A, S287G, A328W, F329M, and Y332G (wherein the amino acid positions are numbered with respect to SEQ ID NO: 6). In one embodiment the variant of human BChE comprises a peptide of SEQ ID NO: 6 modified to comprise each of the amino acid substitutions selected from A199S, S226A, S287G, A328W, F329M, and Y332G (wherein the amino acid positions are numbered with respect to SEQ ID NO: 6). In accordance with one embodiment a butyrylcholinesterase (BChE) enzyme is provided that inactivates the signaling hormone ghrelin and comprises the amino acid sequence of SEQ ID NO: 5 modified to comprise one or more amino acid substitutions selected from H117G, N282Y, H283G, M284T, L285P and any combinations thereof, and one or more amino acid substitutions selected from A199S, S226A, S287G, A328W, F329M, Y332G and any combination thereof, optionally wherein the butyrylcholinesterase (BChE) enzyme comprises the amino acid sequence of SEQ ID NO: 4 modified to comprise one or more amino acid substitutions selected from A227S, S254A, S315G, A356W, F357M, Y3360G and any combination thereof. In accordance with one embodiment a butyrylcholinesterase (BChE) enzyme is provided that inactivates the signaling hormone ghrelin and comprises the amino acid sequence of SEQ ID NO: 4. In one embodiment a polynucleotide is provided that encodes any of the modified butyrylcholinesterase (BChE) enzymes disclosed herein. In one embodiment a polynucleotide is provided that encodes a peptide of SEQ ID NO: 5 modified to comprise one or more amino acid substitutions selected from G117H, Y282N, G283H, T284M, and P285L, and optionally one or more amino acid substitutions selected from A199S, S226A, S287G, A328W, F329M, and Y332G (wherein the amino acid positions are numbered with respect to SEQ ID NO: 5). In one embodiment a polynucleotide is provided that encodes a peptide of SEQ ID NO: 5 modified to comprise two more amino acid substitutions selected from G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a polynucleotide is provided that encodes a peptide of SEQ ID NO: 5 modified to comprise three more amino acid substitutions selected from G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment a polynucleotide is provided that encodes a peptide of SEQ ID NO: 5 modified to comprise four or more amino acid substitutions selected from G117H, Y282N, G283H, T284M, and P285L, and one or more amino acid substitutions selected from A199S, S226A, S287G, A328W, F329M, and Y332G (wherein the amino acid positions are numbered with respect to SEQ ID NO: 5). In one embodiment the polynucleotide encodes a peptide of SEQ ID NO: 6.

In one embodiment the polynucleotide encodes a peptide of SEQ ID NO: 6 modified to comprise one or more amino acid substitutions selected from A199S, S226A, S287G, A328W, F329M, and Y332G (wherein the amino acid positions are numbered with respect to SEQ ID NO: 6). In one embodiment the polynucleotide encodes a peptide of SEQ ID NO: 6 modified to comprise two or more amino acid substitutions selected from A199S, S226A, S287G, A328W, F329M, and Y332G (wherein the amino acid positions are numbered with respect to SEQ ID NO: 6). In one embodiment the polynucleotide encodes a peptide of SEQ ID NO: 6 modified to comprise three more amino acid substitutions selected from A199S, S226A, S287G, A328W, F329M, and Y332G (wherein the amino acid positions are numbered with respect to SEQ ID NO: 6). In one embodiment the polynucleotide encodes a peptide of SEQ ID NO: 6 modified to comprise four or more amino acid substitutions selected from A199S, S226A, S287G, A328W, F329M, and Y332G (wherein the amino acid positions are numbered with respect to SEQ ID NO: 6). In one embodiment the polynucleotide encodes a peptide of SEQ ID NO: 6 modified to comprise each of the amino acid substitutions selected from A199S, S226A, S287G, A328W, F329M, and Y332G (wherein the amino acid positions are numbered with respect to SEQ ID NO: 6).

In one embodiment any of the polynucleotides encoding BChE as disclosed herein can be operably linked to regulatory elements that allow for the expression of the polynucleotide in mammalian cells, including for example, human cells. In one embodiment an expression cassette is provided wherein the expression cassette comprises a nucleic acid sequence that encodes a peptide of SEQ ID NO: 4, SEQ ID NO: 6 or SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G wherein the polynucleotide is operably linked to a promoter that functions in mammalian cells. In one embodiment the promoter is a Cytomegalovirus (CMV) promoter, optionally paired with a tetracycline regulatory element (TRE). In one embodiment a plasmid is provided comprising the expression cassette. In one embodiment an episome is provided comprising the expression cassette. In one embodiment a nucleic acid construct is provided comprising a nucleic acid sequence encoding a BChE peptide of the present invention and a fluorescent marker wherein the BChE and fluorescent marker encoding sequences are both operably linked to an inducible marker. In one embodiment a construct is provided comprising a a Cytomegalovirus (CMV) promoter, optionally paired with a tetracycline regulatory element (TRE) that is operably linked to a nucleic acid sequence encoding:
i) SEQ ID NO: 4 or SEQ ID NO: 6, or an amino acid sequence of SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof; and
ii) a Green Fluorescent Protein (GFP) or a Red fluorescent protein (RFP), optionally wherein the RFP is selected from tdTomato, mCherry, mStrawberry, and DsRed.
   In one embodiment a composition is provided comprising a delivery vehicle and any of the BChE encoding nucleic acid constructs disclosed herein. In one embodiment the delivery vehicle is associated with a construct comprising a sequence that expresses a novel BChE of the present disclosure. In one embodiment an episomal construct is provided that can be delivered into a host cell wherein the episomal construct associates with the nuclear scaffolding and replicates with the cell replication but does not incorporate into the genome. Typically, the episomal construct will replicate once with cell replication, thus extending the duration of expression of the encoded BChE protein. The episomal construct encoding the BChE protein can be further provided with a detectable marker to identify cells carrying the episomal construct. In one embodiment the detectable marker is a fluorophore. The detectable marker gene can be linked to a constitutive promoter or an inducible promoter. In one embodiment the episomal construct comprises a first detectable marker operably linked to a constitutive promoter and an inducible promoter operably linked to a nucleic acid encoding a BChE protein of the present invention, optionally wherein the episomal construct further comprises a second detectable marked linked to the inducible promoter. In one embodiment the first and second detectable markers are different fluorophores, optionally wherein the first and second fluorophores are selected from a Green Fluorescent Protein (GFP) and a Red Fluorescent Protein (RFP).

In one embodiment the delivery vehicle is associated with an episomal construct comprising a Cytomegalovirus (CMV) promoter linked to a first fluorophore (e.g., a Green Fluorescent Protein (GFP)), paired with a tetracycline regulatory element (TRE) that is operably linked to a nucleic acid sequence encoding a BChE peptide of the present invention and a second fluorophore (e.g., a Red Fluorescent Protein (RFP)). As used herein the designation that the nucleic construct is “associated with” the delivery vehicle defines any condition wherein the nucleic acid sequence is bound to, integrated/intercalated with, or encapsulated by, the delivery vehicle. The association can result from any chemical bonding including for example, ionic, covalent, and hydrogen bonding or an electrostatic interaction. In one embodiment the delivery vehicle is a viral vector, including for example an adenovirus vector, a lentivirus viral vector, or an adeno-associated viral vector. In another embodiment the delivery vehicle is a non-viral polymeric nanoparticle delivery vehicle. In one embodiment the delivery vehicle can be selected or modified to comprise a targeting moiety that directs the delivery vehicle to a specific tissue or organ upon administration to a mammal.

In accordance with one embodiment a method of inactivating ghrelin in the cells of a subject is provided. The method comprises the step of administering a composition, comprising a delivery vehicle that is associated with any of the BChE encoding polynucleotides disclosed herein, to the subject in need of a reduction in ghrelin activity, resulting in the intracellular contact of n-octanyl acylated ghrelin with an effective amount of a BChE enzyme to cleave the n-ocanoyl group and inactivate ghrelin. In one embodiment the delivery vehicle is associated with a polynucleotide encoding a BChE enzyme comprising an amino acid sequence of SEQ ID NO: 4, SEQ ID NO: 6 or SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G. In one embodiment the delivery vehicle comprises a polynucleotide encoding a BChE enzyme comprising an amino acid sequence of SEQ ID NO: 6 or SEQ ID NO: 6 further modified by one mor more amino acid substitutions selected from the group consisting of A199S, S226A, S287G, A328W, F329M, and Y332G. In one embodiment the delivery vehicle comprises a polynucleotide encoding a BChE enzyme comprising an amino acid sequence of SEQ ID NO: 4 or SEQ ID NO: 4 further modified by one mor more amino acid substitutions selected from the group consisting of A199S, S226A, S287G, A328W, F329M, and Y332G. In one embodiment the delivery vehicle comprises a polynucleotide encoding a BChE enzyme comprising an amino acid sequence of SEQ ID NO: 6 modified to comprise a F329M substitution optionally with a combination with one or more of the following amino acid substitutions: A199S, F227A, S287G, A328W, or Y332G.

In accordance with one embodiment a method of suppressing drug seeking behavior in a mammal is provided, wherein the method comprising decreasing Ghrelin activity in the cells of the mammal. In one embodiment the method comprises administering a polynucleotide to the mammal, wherein the polynucleotide encodes a polypeptide selected from the group consisting of SEQ ID NO: 2, SEQ ID NO: 4, SEQ ID NO: 6 or a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G. In one embodiment a composition is administered to inhibit drug seeking behavior in an animal wherein the composition comprises a delivery vehicle associated with polynucleotide encodes a polypeptide selected from the group consisting of SEQ ID NO: 2, SEQ ID NO: 4, SEQ ID NO: 6 or a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G, wherein the polynucleotide is operably linked to a promoter that functions in mammalian cells, optionally wherein the promoter is an inducible promoter. In one embodiment, the polynucleotide is operably linked to an inducible promoter, wherein the inducible promoter comprises a tetracycline regulatory element (TRE).

In one embodiment, the composition comprises a delivery vehicle associated with a polynucleotide, wherein the polynucleotide comprises a Cytomegalovirus (CMV) promoter operably linked to a nucleic acid sequence encoding Green Fluorescent Protein (GFP), wherein the polynucleotide further comprises a tetracycline regulatory element (TRE), wherein the TRE is operably linked to a nucleic acid sequence encoding:

SEQ ID NO: 4 or SEQ ID NO: 6, or an amino acid sequence of SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof. In one embodiment the polynucleotide associated with the delivery vehicle further comprises a Red fluorescent protein (RFP) operably linked to said TRE, optionally wherein the RFP is selected from tdTomato, mCherry, mStrawberry, and DsRed. In one embodiment the subject being treated for suppressing drug seeking behavior is a human, and the the drug seeking behavior to be suppressed is narcotic seeking behavior, optionally wherein the narcotic is an opioid. In one embodiment the subject being treated for suppressing drug seeking behavior is a human, and the the drug seeking behavior to be suppressed is cocaine, alcohol or nicotine seeking behavior.

In accordance with one embodiment the delivery vehicle used for the administration of compositions comprising BChE encoding polynucleotides and be any of the viral or non-viral delivery vehicles known to those skilled in the art. In one embodiment the delivery vehicle is a viral vector, including for example an adenovirus vector or an adeno-associated viral vector. In another embodiment the delivery vehicle is a non-viral polymeric nanoparticle delivery vehicle. In one embodiment the delivery vehicle is a viral vector, including for example an adenovirus vector or an adeno-associated viral vector. In another embodiment the delivery vehicle is a non-viral polymeric nanoparticle delivery vehicle. Compositions comprising the delivery vehicle, the BChE encoding polynucleotide and a pharmaceutically acceptable carrier can be administered to a subject using any of the standard techniques, including oral, intravenous, intramuscular, subcutaneous or transdermally.

### EXEMPLARY EMBODIMENTS

In accordance with embodiment 1, a recombinant polynucleotide that expresses a butyrylcholinesterase (BChE) enzyme that inactivates the signaling hormone ghrelin is provided, wherein the polynucleotide encodes a butyrylcholinesterase (BChE) enzyme comprising the amino acid sequence of SEQ ID NO: 2, SEQ ID NO: 4, SEQ ID NO: 6 or the amino acid sequence of SEQ ID NO: 5 modified to comprise one or more amino acid substitutions selected from H117G, N282Y, H283G, M284T, L285P and any combinations thereof, optionally with one or more amino acid substitutions selected from A199S, S226A, S287G, A328W, F329M, Y332G and any combination thereof, optionally wherein the butyrylcholinesterase (BChE) enzyme comprises the amino acid sequence of SEQ ID NO: 6 modified to comprise one or more amino acid substitutions selected from A199S, S226A, S287G, A328W, F329M, Y332G and any combination thereof, optionally wherein the polynucleotide encodes a polypeptide of SEQ ID NO: 4 or SEQ ID NO: 4 modified to comprise one or more amino acid substitutions selected from A227S, S254A, S315G, A356W, F357M, and Y360G.

In accordance with embodiment 2, a plasmid or episome is provided comprising the recombinant polynucleotide of embodiment 1 wherein the polynucleotide is operably linked to a promoter that functions in mammalian cells. In one embodiment the recombinant polynucleotide of embodiment 1 is part of an episomal construct, wherein the recombinant polynucleotide is operably linked to a promoter that functions in mammalian cells, optionally wherein the promoter is an inducible promoter.

In accordance with embodiment 3, the recombinant polynucleotide according to embodiment 1 or 2 is provided wherein the promoter is an inducible promoter.

In accordance with embodiment 4, the recombinant polynucleotide according to any one of embodiments 1-3 is provided wherein the recombinant polynucleotide comprises a tetracycline regulatory element (TRE) that is operably linked to the polynucleotide encoding the BChE enzyme, optionally wherein the recombinant polynucleotide is formed as a plasmid or an episome.

In accordance with embodiment 5, the recombinant polynucleotide of any one of embodiments 1˜4 is provided wherein said recombinant polynucleotide comprises a Cytomegalovirus (CMV) promoter operably linked to a tetracycline regulatory element (TRE), wherein the TRE is operably linked to a nucleic acid sequence encoding: SEQ ID NO: 4 or SEQ ID NO: 6, or an amino acid sequence of SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof.

In accordance with embodiment 6, the recombinant polynucleotide of any one of embodiments 1-5 is provided, further comprising
a Green Fluorescent Protein (GFP) or a Red fluorescent protein (RFP) operably linked to a tetracycline regulatory element (TRE) that is operably linked to the polynucleotide encoding the BChE enzyme, optionally wherein the RFP is selected from tdTomato, mCherry, mStrawberry, and DsRed.

In accordance with embodiment 7, a recombinant polynucleotide is provided comprising:
i) an inducible promoter that is functional in mammalian cells; and
ii) a nucleic acid sequence encoding a BChE peptide, said BChE peptide encoding nucleic acid sequence being operably linked to said inducible promoter, wherein the BChE peptide comprises the amino acid sequence of SEQ ID NO: 4, SEQ ID NO: 6 or the amino acid sequence of SEQ ID NO: 5 modified to comprise one or more amino acid substitutions selected from H117G, N282Y, H283G, M284T, L285P and any combinations thereof, and one or more amino acid substitutions selected from A199S, S226A, S287G, A328W, F329M, Y332G and any combination thereof, optionally wherein the butyrylcholinesterase (BChE) enzyme comprises the amino acid sequence of SEQ ID NO: 6 modified to comprise one or more amino acid substitutions selected from A199S, S226A, S287G, A328W, F329M, Y332G and any combination thereof, optionally wherein the polynucleotide encodes a polypeptide of SEQ ID NO: 4 or SEQ ID NO: 4 modified to comprise one or more amino acid substitutions selected from A227S, S254A, S315G, A356W, F357M, and Y360G optionally wherein said recombinant polynucleotide is formed as a plasmid or an episome.

In accordance with embodiment 8, the recombinant polynucleotide of embodiment 7 is provided wherein the inducible promoter comprises a tetracycline regulatory element (TRE) that is operably linked to said nucleic acid sequence encoding said BChE enzyme.

In accordance with embodiment 9, the recombinant polynucleotide of embodiment 7 or 8 is provided wherein the inducible promoter comprises a Cytomegalovirus (CMV) promoter operably linked to a tetracycline regulatory element (TRE).

In accordance with embodiment 10 a composition is provided comprising the plasmid of any one of embodiments 1 to 9; and a viral delivery vehicle wherein the recombinant polynucleotide is associated with the viral delivery vehicle.

In accordance with embodiment 11 the composition of embodiment 10 is provided wherein the viral delivery vehicle is an adeno-associated viral vector.

In accordance with embodiment 12 the composition of any one of embodiments 10-11 is provided wherein a capsid protein of the viral delivery vehicle has been modified to comprise a targeting moiety.

In accordance with embodiment 13 a composition is provided comprising the recombinant polynucleotide of any one of embodiments 1 to 9; and a non-viral delivery vehicle, wherein the recombinant polypeptide is associated with the non-viral delivery vehicle, optionally wherein the non-viral delivery vehicle is a Lipid Nanoparticles (LNPs), Polymer Nanoparticles (PNPs), Extracellular Vesicles (EVs), for example an exosome, polyplexes formed with cationic polymers (e.g., Poly(ethyleneimine) (PEI)), liposomes.

In accordance with embodiment 14 the composition of embodiment 13 is provided wherein the non-viral delivery vehicle is a polymeric nanoparticle.

In accordance with embodiment 15 a composition of embodiment 14 is provided wherein the non-viral polymeric nanoparticle delivery vehicle is covalently linked to a targeting moiety.

In accordance with embodiment 16 a composition of any one of embodiments 13-15 is provided wherein the non-viral polymeric nanoparticle delivery vehicle comprises a nucleic acid sequence, optionally wherein the nucleic acid is an origami nucleic acid sequence.

In accordance with embodiment 17 a composition of embodiment 14 or 15 is provided wherein the non-viral polymeric nanoparticle delivery vehicle comprises a raft copolymer.

In accordance with embodiment 18 a method of suppressing drug seeking behavior in a mammal is provided, wherein the method comprises administering a polynucleotide to the mammal, wherein the polynucleotide encodes a butyrylcholinesterase (BChE) polypeptide selected from the group consisting of SEQ ID NO: 2, SEQ ID NO: 4 or a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G, optionally wherein the butyrylcholinesterase (BChE) polypeptide comprises the amino acid sequence of SEQ ID NO: 6 or SEQ ID NO: 6 modified to comprise one or more amino acid substitutions selected from A199S, S226A, S287G, A328W, F329M, Y332G and any combination thereof.

In accordance with embodiment 19 a method of reducing body weight within a mammal, reducing body weight gain in a mammal, reducing a mammal's level of aggression, reducing a mammal's response to cocaine, and/or reducing the severity of a stress-induced condition is provided wherein the method comprises administering a polynucleotide to the mammal, wherein the polynucleotide encodes a butyrylcholinesterase (BChE) polypeptide selected from the group consisting of SEQ ID NO: 2, SEQ ID NO: 4 or a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G, optionally wherein the butyrylcholinesterase (BChE) polypeptide comprises the amino acid sequence of SEQ ID NO: 6 modified to comprise one or more amino acid substitutions selected from A199S, S226A, S287G, A328W, F329M, Y332G and any combination thereof.

In accordance with embodiment 20 the method of embodiment 18 or 19 is provided wherein the mammal is a human.

In accordance with embodiment 21 the method of embodiment 18 or 20 is provided wherein the drug seeking behavior to be suppressed is narcotic seeking behavior, optionally wherein the narcotic is an opioid.

In accordance with embodiment 22 the method of embodiment 18 or 19 is provided wherein the drug seeking behavior to be suppressed is cocaine seeking behavior.

In accordance with embodiment 23 the method of embodiment 18 or 19 is provided wherein the drug seeking behavior to be suppressed is alcohol seeking behavior.

In accordance with embodiment 24 the method of embodiment 18 or 19 is provided wherein the drug seeking behavior to be suppressed is nicotine seeking behavior.

In accordance with embodiment 25 the method of any one of embodiments 18-24 is provided, wherein the polynucleotide encodes a polypeptide of SEQ ID NO: 4 or a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G, optionally wherein the polynucleotide encodes a polypeptide of SEQ ID NO: 6 or SEQ ID NO: 6 modified to comprise one or more amino acid substitutions selected from A199S, S226A, S287G, A328W, F329M, and Y332G, optionally wherein the polynucleotide encodes a polypeptide of SEQ ID NO: 4 or SEQ ID NO: 4 modified to comprise one or more amino acid substitutions selected from A227S, S254A, S315G, A356W, F357M, and Y360G.

In accordance with embodiment 26 the method of any one of embodiments 18-24 is provided, wherein the polynucleotide encodes a polypeptide comprising SEQ ID NO: 6 modified to comprise a F329M substitution optionally in combination with one or more of the following amino acid substitutions: A199S, F227A, S287G, A328W, or Y332G.

In accordance with embodiment 27 the method of any one of embodiments 18-26 is provided, wherein the polynucleotide is administered using a viral or non-viral delivery vehicle.

In accordance with embodiment 28, a method of inactivating ghrelin is provided wherein the method comprises the step of contacting n-octanyl acylated ghrelin with an effective amount of a BChE enzyme to cleave the n-ocanoyl group and therefore inactivating ghrelin, the BChE enzyme comprising an amino acid sequence of SEQ ID NO: 4, SEQ ID NO: 6 or modified SEQ ID NO: 5, wherein the modification to SEQ ID NO: 5 comprise one or more amino acid substitutions selected from G117H, Y282N, G283H, T284M, and P285L, and one or more amino acid substitutions selected from A199S, S226A, S287G, A328W, F329M, and Y332G.

In accordance with embodiment 29, the method of embodiment 28 is provided wherein the BChE enzyme comprises an amino acid sequence of SEQ ID NO: 6, or SEQ ID NO: 6 modified to comprise one or more amino acid substitutions selected from A199S, S226A, S287G, A328W, F329M, and Y332G or SEQ ID NO: 4 or SEQ ID NO: 4 modified to comprise one or more amino acid substitutions selected from A227S, S254A, S315G, A356W, F357M, and Y360G.

In accordance with embodiment 30, the method of embodiment 28 is provided wherein the BChE enzyme comprises an amino acid sequence of SEQ ID NO: 6 modified to comprise a F329M substitution optionally with a combination with one or more of the following amino acid substitutions: A199S, F227A, S287G, A328W, or Y332G.

The polymer nanoparticles described herein are capable of interacting with (e.g., encapsulating or complexing with) polynucleotides, including nucleotide plasmids and episomes. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency is about 80% to about 95%.

The compositions described herein can be used for treating subjects in need of a reduction in ghrelin activity. For example, a polymer nanoparticle may be able to deliver a nucleic acid (i.e., a payload) that provides a therapeutic benefit to a patient.

In some embodiments, a polymer nanoparticle comprises a block copolymer according to the present disclosure. In some embodiments, the block copolymer self-assembles into the nanoparticle.

In some embodiments, a composition comprises a polymer nanoparticle as described herein and a nucleic acid (sometimes called a payload) complexed to the polymer nanoparticle. For example, the nucleic acid may be complexed to the nanoparticle through electrostatic interactions. In certain embodiments, the polymer nanoparticle of the composition serves as a transfection agent to deliver a nucleic acid to a cell.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

In certain embodiments, a block copolymer comprises a first block and a second block, the first block may comprise a homopolymer of poly dimethylaminoethyl methacrylate (DMAEMA), and the second block may comprise a homopolymer of poly butyl methacrylate (BMA). For example, in one embodiment, the first block comprises 100% DMAEMA and the second block comprises 60% BMA and 40% PAA, with the final copolymer comprising 80% block 1 and 20% block 2. The overall theoretical molecular weight was 43.905 KDa.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### EXAMPLES

### Example 1

In Vitro Transfection of Human Embryonic Kidney (HEK) Cells with Butyrylcholinesterase

The following experiments are directed to determining optimal parameters for plasmid delivery into HEK293T cells and to assess their transfection efficiency.

Cells were plated on day 1 at both 25,000 and 35,000 cells per well in a 96 well tissue culture treated plate. Media only wells were added around the edge of the plate to avoid edge effect evaporation skewing sample data. The plate was incubated at 37° C., 5% CO₂ overnight.

Each plasmid was tested in duplicate at both a 0.1 μg concentration and 0.2 μg for each seeding density. Controls for this experiment included a non-treatment control (NTC), Lipofectamine only, 0.1 μg pDNA, and 0.2 μg pDNA. Dosing occurred 24 hrs after plating (day 2), which allowed cell growth to reach 70-80% confluency. The Lipofectamine treatments were individually made in tubes following vendor guidelines and incubated at room temperature for 5 min. After incubation 10 μl of the appropriate treatment was added to each well and the plate was placed back in an incubator to allow time for transfection.

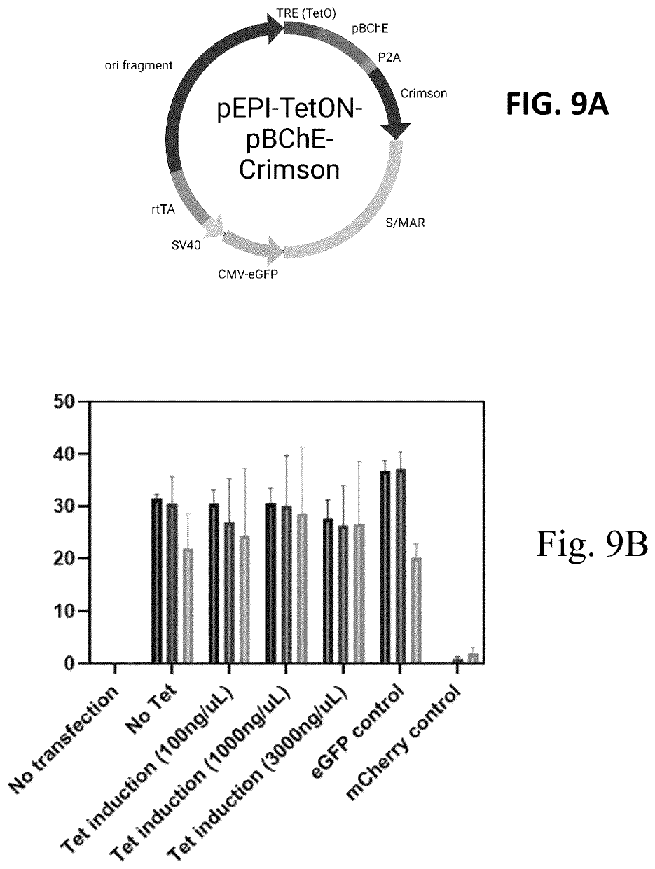

Images were taken at both 24 hours and 48 hours post dosing to give an estimate on viability and transfection efficiency. The images indicated Native Human and Porcinated Human were the best performing plasmids with transfection efficiencies at 72% and 89% respectively (FIG. 7B) with the best seeding density at 25,000 and a 0.2 μg dosing concentration.

After 48 hours (day 4), the cells went through a staining and fixing process to be analyzed on a flow cytometer, to confirm results seen in imaging and give accurate percentages of viability and transfection efficiency. Preparation of the plates comprises removal of the cell growth media, followed by washing with Phosphate-buffered saline (PBS) and addition of 0.25% Trypsin-EDTA. The plates were then placed in an incubator at 37° C., 5% CO₂ until cells detached. Following detachment, cell growth media was added to deactivate trypsin, and the cell suspensions were pipetted to a tube, and centrifuged at 300×g for 5 minutes to pellet cells. During centrifugation, a 1:1,000 Zombie Violet viability dye was prepared for staining following vendor guidelines. Following centrifugation, the supernatant was removed, and the cells were washed with PBS. Shortly, the PBS was removed and replaced with the prepared Zombie Violet dye and mixed with the pelleted cells. The plate was covered in foil, and incubated at room temperature for 20 minutes in the dark. Following the incubation, the plates were centrifuged at 300×g for 5 minutes, and the supernatant removed, followed by washing by PBS. The PBS was then removed and replaced with 4% Paraformaldehyde (PFA) and allowed to incubate at room temperature for 10 minutes in the dark. The supernatant was removed, and the cells were resuspended in PBS, sealed, and placed at 4° C. until analysis.

The samples were then run on the cytometer 4 days later and showed transfection percentage for the top two plasmids at 72.7% and 77.4% for native human and porcinated human butyrylcholinesterase (BChE), respectively.

### Example 2

Transfection into Cells with Fluorescent Reporters Demonstrating Successful Transfection and Expression of the BChE

The following experiments were conducted to assess the the baseline transfection capabilities of the pEPI-TetON-pBChE-Crimson episome by way of eGFP fluorescence and the induction efficiency of the tetracycline response element (TRE) by way of Crimson (red) fluorescence.

The Lipofectamine treatments were individually made in tubes following vendor guidelines and incubated at room temperature for 5 min. After incubation 10 μl of the appropriate treatment was added to each well. It was then prepped for dosing in individual tubes with one condition being 0.3 μg of pDNA and lipofectamine. Then, samples were incubated at room temperature for 5 min before 10 μl of the appropriate treatment was added to each well. The plate was placed back in an incubator to allow time for transfection to occur.

Images were taken at 24, 36, and 48 hr post dosing to give an estimate on transfection efficiency and expression of target fluorescence.

Doxycycline at different concentrations was added to wells (100, 1000, or 3000 ng/ul) 24 hours after addition of the DNA construct, then imaged 12 hours and 24 hours later (corresponding to 24 hr, 36 hr, and 48 hr after transfection delivery).

### Example 3

### Quantification of BChE and Ghrelin Following BChE Transfection

### Quantification of BChE and Ghrelin Following BChE Transfection Via ELISA

HEK293T cells were plated on day 1 at 25,000 cells/well on a 96 well tissue culture treated plate. Media only wells were added around the edge of the plate to avoid edge effect evaporation skewing any sample data.

Each plasmid was tested in triplicate at a 0.2 μg concentration for each time point, 4, 2, and 1 hours. Controls for this experiment included a Cytomegalovirus (CMV) GFP plasmid treatment and cell growth media only wells. Dosing occurred 24 hrs after plating (day 2), which allowed cell growth to reach 70-80% confluency. The Lipofectamine treatments were individually made in tubes following vendor guidelines and incubated at room temperature for 5 minutes. After incubation 10 μl of the appropriate treatment was added to each well and the plate was placed back in an incubator to allow time for transfection to occur.

Images were taken at 48 hour post dosing to give an estimate of transfection efficiency. This showed rather low, yet similar, transfection efficiencies for all plasmids, averaging around 21%.

72 hours post dosing (day 5), the media was collected into a separate plate to be sent for BChE ELISA analysis. With the remaining cells, the 1- and 2-hour wells were refilled with 120 μl of cell growth media. For the 4 hour wells, 120 μl of a Ghrelin/cell growth media mixture equaling a 250 μg/ml was added and the plate was placed back in an incubator. After 2 hours, the wells labeled ‘2 hour’ the media was removed and 120 μl of the Ghrelin/cell growth media mixture added. The same was done for the ‘1 hour’ wells after an additional hour of incubation passed. After 4 hours from the initial Ghrelin addition, the media was collected into a separate plate to be sent for Ghrelin ELISA analysis. The BChE ELISA showed quantifiable BChE levels, with native human and porcinated human the best performing.

### Quantification of BChE and Ghrelin Following BChE Transfection Via ELISA

We measured the amount of BChE enzyme produced 72 hours post transfection as well as the remaining amount of activated human ghrelin after incubation with BChE enzyme for 4, 2 and 1 hours. The data was re-run performed to replicate the previous ELISA experiment where BChE and ghrelin was quantified following BChE transfection.

HEK293T cells were plated on day 1 at 25,000 cells per well on a 96 well tissue culture treated plate. Media only wells were added around the edge of the plate to avoid edge effect evaporation.

Each plasmid was tested in triplicate at a 0.2 μg concentration for each time point, 4, 2, and 1 hours. Controls for this experiment included a CMV GFP plasmid and cell growth media only wells. Dosing occurred 24 hours after plating (day 2), which allowed cell growth to reach 70-80% confluency. The Lipofectamine treatments were individually made in tubes following vendor guidelines and incubated at room temperature for 5 minutes. After incubation 10 μl of the appropriate treatment was added to each well and the plate was placed back in an incubator to allow time for transfection to occur.

Images were taken at 48 hour post dosing to estimate transfection efficiency. This method showed higher transfection efficiency than the previous experiment, with most plasmids averaging around 40% transformation efficiency with porcinated human BChE having an average of 67.5%.

72 hours post dosing (day 5), the media was collected into a separate plate to be sent for BChE ELISA analysis. With the remaining cells, the 1- and 2-hour wells were refilled with 120 μl of cell growth media. For the 4 hour wells, 120 μl of a Ghrelin/cell growth media mixture equaling a 250 μg/ml was added and the plate was placed back in an incubator. After 2 hours, the wells labeled ‘2 hour’ had the media removed and 120 μl of the ghrelin/cell growth media mixture added. The same was done for the ‘1 hour’ wells after an additional hour of incubation had passed. After 4 hours from the initial ghrelin addition, the media was collected into a separate plate to be sent for analysis.

The ELISA confirmed that native human and porcinated human BChE showed high production. The ghrelin ELISA showed administration of all plasmids resulted in a reduction of ghrelin over time with the lowest value from the 4 hour incubation, with BChE plasmids showing a greater reduction at early timepoints.

### Example 4

### PNP1 Preparation

### Polymer Nanoparticle Synthesis

PNP1 was synthesized similarly as described in U.S. Patent Application Publication No. 2022/0175812, the entirety of which is incorporated by reference herein, with some modifications using reversible addition-fragmentation chain transfer (RAFT) polymerization with reagents and amounts listed in Table 1. PNP1 is a diblock copolymer where the first block is p-DMAEMA and the second block is p-BMA.

TABLE 1
Monomers used in the nanoparticle forming polymers (PNPs).

| Monomer Name | Abbreviation | CAS | Structure |
|---|---|---|---|
| Butyl methacrylate | BMA | 97-88-1 | 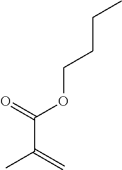 |
| Dimethylaminoethyl methacrylate | DMAEMA | 2867-47-2 | 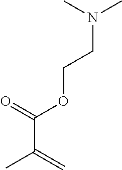 |

### Example 5

Transfection with Carriers: Comparison

The basis of this experiment was to quantify the amount of BChE produced 72 hours post transfection and the remaining amount activated human ghrelin after incubation with BChE for 4, 2 and 1 hours using polymer nanoparticles (PNPs) compared to Lipofectamine 3000 as a carrier.

Two plates of HEK293T cells were seeded on day 1 at 25,000 cells per well on 96 well tissue culture treated plates. Each plasmid was tested in duplicate at a 0.2 μg concentration for each time point, 4, 2, and 1 hours and each carrier, Lipofectamine, and PNP1, and PNP2 on both plates. A control for this experiment included a CMV GFP plasmid. Dosing occurred 24 hours after plating (day 2), which allowed cell growth to reach 70-80% confluency. Composition of PNPs tested=p (DMAEMA)-b-p (BMA-co-PAA).

The Lipofectamine treatments were made individually in tubes following vendor guidelines and incubated at room temperature for 5 minutes. After incubation, 10 μl of each treatment were added to the wells. Both PNP stocks were sonicated for 10 minutes prior to treatment preparation. PNP1 was then prepared for dosing in individual tubes at concentration of 0.2 μg pDNA and 0.02 mg/mL PNP, incubated at room temperature for 5 min prior to addition of 10 μl of the appropriate treatment added to each well. The plate was placed back in an incubator to allow for transfection.

Images were taken at 48 hours post dosing to give an estimate of transfection efficiency. The results indicated an average 12% transfection with Lipofectamine and 1-4% for PNP1. PNP1 had 4% transfection and low cell toxicity The PNP1 composition is a p (DMAEMA) BMA polymeric nanoparticle formed from the following components: Block 1: p (DMAEMA) and Block 2: BMA.

72 hours post dosing (day 5), the media in plate 1 was collected in a separate plate to be sent for BChE ELISA analysis. For the second plate, the 4 hour wells had 1.1 μl of Ghrelin/cell growth media mixture at a concentration of 250 μg/mL added before the plate was placed back in an incubator. No media was removed. After 2 hours, the wells labeled ‘2 hour’ had 1.1 μl of a Ghrelin/cell growth media mixture added. The same was done for the ‘1 hr’ wells following additional hour of incubation. After 4 hours, the media was collected into a separate plate to be sent for Ghrelin ELISA analysis.

The BChE ELISA showed high BChE production levels, and the activated ghrelin ELISA demonstrated reduced levels of activated ghrelin following incubation in the BChE producing wells.

Transfection with PNPs for Ghrelin

This experiment was to quantify the remaining amount of activated human ghrelin after an incubation with the BChE enzyme for 90, 60, and 30 minutes. This was run with shorter time points to be comparable to the ghrelin assay.

HEK293T cells were plated on day 1 at 25,000 cells per well on a 96 well tissue culture treated plate. Each plasmid was tested in duplicate at a concentration of 0.2 μg for each time point (90, 60, and 30 minutes) and each carrier (Lipofectamine and PNP1). The control for this experiment was a CMV GFP plasmid. Dosing occurred 24 hours after plating (day 2), which allowed cell growth to reach 70-80% confluency.

The Lipofectamine treatments were individually made in tubes following vendor guidelines and incubated at room temperature for 5 minutes. After incubation 10 μl of the appropriate treatment was added to each well. The PNP stock was sonicated for 10 min prior to treatment preparation. The stock was then prepared for dosing in individual tubes with at a concentration of 0.2 μg pDNA and 0.02 mg/ml PNP. Then, each were incubated at room temperature for 5 minutes before 10 μl of the appropriate treatment added to each well. The plate was placed back in an incubator to allow time for transfection.

Images were taken at 48 hours post dosing to give an estimate of transfection efficiency. The images indicated an average 20% transfection for Lipofectamine and 14% of the PNP.

72 hours post dosing (day 5), the 90 minute wells had 1.1 μl of a Ghrelin/cell growth media mixture equaling 250 μg/mL added and the plate placed back in an incubator. No media was removed. After 30 minutes, the wells labeled ‘60 minute’ had 1.1 μl of a ghrelin/cell growth media mixture added. The same was done for the ‘30 minute’ wells after an additional 30 min of incubation had passed. After 90 min from the initial ghrelin addition, the media was collected into a separate plate to be sent for ghrelin ELISA analysis.

The ghrelin ELISA showed both native human and porcinated human BChE successfully lowered activated ghrelin levels in comparison to control. It also showed comparable decrease from both carriers Lipofectamine and the PNP.

### Example 6

### Administration of BChE to Male CD-1 Mice, Age 8-12 Weeks Old

The delivery vehicle used for this experiment was p (DMAEMA)-b-p (BMA-co-PAA). L049-P loaded PNPs (p (DMAEMA)-b-p (BMA-co-PAA)) were administered at 1 mg/kg by IM injection into the right caudal thigh muscle from the dorsal side.

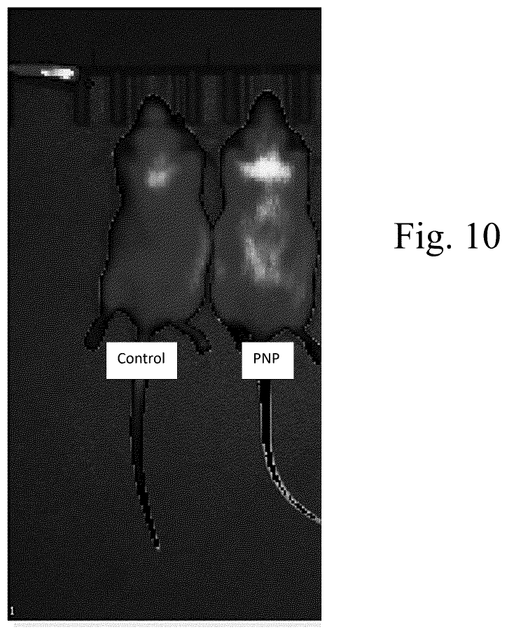

Treated mice were imaged 3 days following injection. For imaging in the In Vivo Imaging System (IVIS). Animals were anesthetized with 3% isoflurane and placed in imaging chamber. Image Acquisition Settings: manual exposure, dorsal and ventral. Excitation/Emission: 488/507. Exemplary results are shown in FIG. 10.

1. A recombinant polynucleotide that expresses a butyrylcholinesterase (BChE) enzyme that inactivates the signaling hormone ghrelin, the polynucleotide comprising
a first nucleic acid sequence that encodes a butyrylcholinesterase (BChE) enzyme, said enzyme comprising the amino acid sequence of SEQ ID NO: 4 or SEQ ID NO: 6, or the amino acid sequence of SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof.

2. The recombinant polynucleotide of claim 1, further comprising a promoter that functions in mammalian cells, wherein said promoter is operably linked to said first nucleic acid sequence.

3. The recombinant polynucleotide of claim 2, wherein said promoter is an inducible promoter.

4. The recombinant polynucleotide of claim 3, wherein said first nucleic acid sequence encodes a polypeptide comprising SEQ ID NO: 6 or a sequence encoding a polypeptide comprising SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof.

5. The recombinant polynucleotide of claim 4, further comprising
a Red fluorescent protein (RFP) operably linked to said inducible promoter and
a constitutive promoter operably linked to a Green fluorescent protein (GFP), optionally wherein the constitutive promoter is a Cytomegalovirus (CMV) promoter.

6. A composition comprising
the recombinant polynucleotide of claim 1 operably linked to a promoter for expressing the BChE enzyme in mammalian cells;
a delivery vehicle, wherein the delivery vehicle is
i) a viral delivery vehicle, selected from the group consisting of adeno-associated viruses, lentiviruses, and adenoviruses; or
ii) a non-viral polymeric nanoparticle delivery vehicle, selected from the group consisting of a Lipid Nanoparticles (LNPs), Polymer Nanoparticles (PNPs), exosomes, an origami nucleic acid, and liposomes; and
a pharmaceutically acceptable carrier, wherein said polynucleotide is associated with the delivery vehicle.

7. The composition of claim 6, wherein the delivery vehicle is covalently linked to a targeting moiety.

8. The composition of claim 6, wherein the delivery vehicle comprises a non-viral polymeric nanoparticle or an origami nucleic acid sequence.

9. The composition of claim 8, wherein the delivery vehicle is a non-viral polymeric nanoparticle delivery vehicle that comprises a raft copolymer.

10. A method of suppressing drug seeking behavior in a mammal, the method comprising administering to the mammal a composition comprising a polynucleotide that encodes a polypeptide selected from the group consisting of SEQ ID NO: 2, SEQ ID NO: 4, SEQ ID NO: 6 and a polypeptide of SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G, wherein said polynucleotide is operably linked to a promoter that functions in mammalian cells;
and a delivery vehicle, wherein the delivery vehicle is associated with said polynucleotide; and
a pharmaceutically acceptable carrier.

11. The method of claim 10 wherein said promoter is an inducible promoter.

12. The method of claim 11 wherein said polynucleotide further comprises a Cytomegalovirus (CMV) promoter operably linked to a nucleic acid sequence encoding a first fluorescent protein, and the inducible promoter comprises a tetracycline regulatory element (TRE), wherein the TRE is operably linked to a nucleic acid sequence encoding:
a polypeptide of SEQ ID NO: 4 or SEQ ID NO: 6, or a polypeptide comprising SEQ ID NO: 5 modified to comprise one or more amino acid substitutions selected from G117H, Y282N, G283H, T284M, and P285L or any combinations thereof, and one or more amino acid substitutions selected from A199S, S226A, S287G, A328W, F329M, and Y332G or any combination thereof.

13. The method of claim 12, further comprising
a Red fluorescent protein (RFP) operably linked to said TRE, wherein the first fluorescent protein is a Green Fluorescent Protein (GFP).

14. The method of claim 11, wherein the mammal is a human and
i) the drug seeking behavior to be suppressed is narcotic seeking behavior, optionally wherein the narcotic is an opioid;
ii) the drug seeking behavior to be suppressed is cocaine seeking behavior;
iii) the drug seeking behavior to be suppressed is alcohol seeking behavior; or
iv) the drug seeking behavior to be suppressed is nicotine seeking behavior.

15. The method of claim 14, wherein the polynucleotide encodes a polypeptide comprising SEQ ID NO: 5 modified to comprise one or more amino acid substitutions selected from G117H, Y282N, G283H, T284M, and P285L, and one or more amino acid substitutions selected from A199S, S226A, S287G, A328W, F329M, and Y332G.

16. The method of claim 14, wherein the polynucleotide encodes a polypeptide comprising SEQ ID NO: 6, or a polypeptide of SEQ ID NO: 6 modified to comprise a F329M substitution optionally with one or more of the following amino acid substitutions: A199S, F227A, S287G, A328W, or Y332G.

17. A method of inactivating ghrelin, the method comprising the step of contacting n-octanyl acylated ghrelin with an effective amount of a BChE enzyme to cleave the n-ocanoyl group and therefore inactivating ghrelin, wherein the BChE enzyme comprises polypeptide of SEQ ID NO: 6 or a polypeptide of SEQ ID NO: 5, wherein the sequence of SEQ ID NO: 5 is modified to comprise one or more amino acid substitutions selected from G117H, Y282N, G283H, T284M, and P285L, and one or more amino acid substitutions selected from A199S, S226A, S287G, A328W, F329M, and Y332G.

18. The method of claim 17, wherein the BChE enzyme comprises the polypeptide of SEQ ID NO: 4 modified to comprise one or more amino acid substitutions selected from A227S, S254A, S315G, A356W, F357M, and Y360G.
